(19) United States
(12) Patent Application Publication
(10) Pub. No.: US 2024/0183760 A1
(43) Pub. Date: Jun. 6, 2024

(54) SYSTEM AND ARRANGEMENT FOR MEASURING A GAS FLOWING IN A GAS LINE

(71) Applicant: Meter-Q Solutions GmbH, Butzbach (DE)

(72) Inventors: Achim ZAJC, Butzbach (DE); Jan SUHR, Butzbach (DE)

(21) Appl. No.: 18/530,463

(22) Filed: Dec. 6, 2023

(30) Foreign Application Priority Data
Dec. 6, 2022 (EP) 22211799.6

(51) Int. Cl. G01N 1/22

(52) U.S. Cl. CPC G01N 1/2247; G01N 2001/2285

(57) ABSTRACT

A system and an arrangement for measuring a gas flowing in a gas line, the system comprising an extraction probe having an inlet opening for extracting a gas flowing in a gas line, an outlet opening for at least partially outputting the extracted gas into the gas line, and an extraction line which fluidically connects at least the inlet opening and the outlet opening, the extraction probe being configured in such a way that at least the inlet opening and the outlet opening are arrangeable in the gas line, the extraction probe being configured in such a way that the outlet opening is arrangeable downstream of the inlet opening along a flow direction of the gas; at least one measuring device for measuring the gas, the extraction line having at least one interface via which the gas is directable at least partially to the measuring device.

### PRIORITY CLAIM FOR FOREIGN APPLICATION

This application claims the benefit of priority under 35 U.S.C. § 119(a) of EP Application No. 22 211 799.6, filed Dec. 6, 2022, the disclosure of which is incorporated by reference herein in its entirety.

### TECHNICAL FIELD

The invention relates to a system and an arrangement for measuring the quality of a gas flowing in a gas line.

### SUMMARY

In gas lines, in particular gas lines of natural gas networks, it is necessary to carry out a measuring of the quality of the gas flowing in the gas line at one or more measuring points. In such gas lines, the gas flows at high velocities and a high pressure in order to enable rapid transport of the gas across the corresponding network. Typical flow velocities may be between 2 to 35 m/s and a typical pressure may be between 5 to 100 barg.

Appropriate measuring devices can be used to measure the quality. These usually have an extraction probe that can be arranged in the gas line and extract gas. If the pressure of the gas in the line is high, the gas in the extraction probe also has a high pressure and, depending on the measuring device, must first be directed via a pressure reducing device to reduce the pressure. Subsequently, the gas is directed to the measuring device, for example, to measure the quality. Typically, only a portion of the gas is used for measuring the quality. The remaining portion as well as the measured gas are discharged to the environment. A return to the gas line is not envisaged, since this is not economical by means known to date. The gas taken from the gas line would first have to be collected and then the pressure of the collected gas would have to be increased in order to be able to feed the gas back into the gas line. This would mean that more units, more installation space and energy would be required. Accordingly, there is a loss of gas in the line on the one hand and an increased environmental impact due to the emission of the gas on the other hand.

It is an objective of the present invention to remedy and/or at least improve one or more of the aforementioned disadvantages. In particular, it is an objective of the present invention to recycle at least a portion of the extracted gas and to reduce the environmental impact by reducing the portion of the gas to be discharged to the environment.

According to a first aspect, the objective is solved by a system for measuring a gas flowing in a gas line, comprising an extraction probe having an inlet opening for extracting a gas flowing in a gas line, an outlet opening for at least partially outputting the extracted gas into the gas line, and an extraction line fluidically connecting at least the inlet opening and the outlet opening. The extraction probe is configured such that at least the inlet opening and the outlet opening are arrangeable in the gas line, the extraction probe being configured such that the outlet opening is arrangeable downstream of the inlet opening along a flow direction of the gas flowing in the gas line. The system further comprises at least one measuring device, the extraction line having an interface through which the gas is at least partially directable to the measuring device for measuring the gas.

The gas in the gas line may be provided at a high pressure and/or flow velocity. The pressure of the gas in the gas line may be between 5 to 100 barg. The flow velocity of the gas in the gas line may be between 2 to 35 m/s. By means of the extraction probe, a “loop” or circuit may be provided in which the gas is extracted from the gas line, particularly at the high pressure and/or flow velocity from the inlet opening, and the gas may be directed to the outlet opening via the extraction line. By means of the interface, a portion of the extracted gas may be extracted from the extraction line and directed to the measuring device for measuring the gas. Accordingly, already at the interface, only a reduced portion of the gas may be extracted and directed to the measuring device or a subsequently defined pressure reducing device for reducing a pressure of the extracted gas. The remaining portion of the gas in the extraction line at high pressure may be directed back to the gas line. Accordingly, the amount of gas that is extracted for measurement is reduced. A further advantage of the solution is that the pressure and/or the flow velocity of the gas in the gas line are used. The arrangement of the inlet and outlet openings in the gas line may cause a pressure difference between the inlet and outlet openings, in particular due to a stagnation pressure at the inlet opening and a dynamic pressure reduction at the outlet opening. This pressure difference may cause the gas to be extracted through the inlet opening, flow through the extraction line, and to the outlet opening, as the pressure difference may cause a flow or flow velocity within the extraction probe. For example, the pressure of the gas within the gas line may be 100 bar, so that there may be a stagnation pressure of 5 bar of the gas at the inlet opening, thus a total pressure of 105 bar at the inlet opening, and a dynamic pressure of 2 bar of the gas at the outlet opening, thus a total pressure of 98 bar at the outlet opening. Thus, there is a pressure difference of 7 bar between the inlet and outlet openings.

Based on the pressure of the gas in the gas line, a subsequently defined flow path and/or a measuring objective of the measuring device, gas may advantageously be extracted from the gas line by means of the extraction probe, partially directed to the measuring device and partially directed to the outlet opening, whereby only an amount of gas necessary for measuring the measuring device is extracted from the extraction line by means of the interface and the remaining portion may be output back into the gas line. Further, based on the pressure of the gas in the gas line, the extracted gas may flow naturally, thus without further means, along a predetermined flow path with a predetermined flow velocity. Accordingly, at least the measuring device may be spaced apart from the gas line. A spacing of the measuring device may advantageously be further increased by providing a pump configured to at least partially provide a predetermined pressure difference in the extraction probe. Accordingly, a distance between the gas line and the device, in particular the pressure reducing device, may be increased without having to accept losses in the measurement or flow velocity of the extracted gas and thus the measurement time.

Upstream and/or downstream may comprise and/or denote a position of a unit and/or component of the system along a flow of the gas. The gas flows along the flow direction in the gas line. Further, the gas may flow along a line, such as the extraction line from the inlet opening to the outlet opening, wherein the direction of the gas flowing in the extraction line may be different from the flow direction. The extraction probe may be configured such that the outlet opening is arrangeable downstream, in particular downstream of the inlet opening, along the flow direction of the gas, in particular of the gas flowing in the gas line, and the inlet opening is arrangeable upstream, in particular upstream of the outlet opening, with respect to the flow and/or flow direction of the gas in the gas line.

The extraction probe, in particular the inlet opening may be configured and arrangeable in the gas line in such a way that the gas flowing in the gas line is extractable from the gas line by means of the inlet opening due to the flow velocity of the gas.

The extraction probe may be configured such that the pressure difference between a first pressure of the gas extracted by means of the extraction probe at the inlet opening and a second pressure of the gas directed by means of the outlet opening may be generated, wherein the pressure difference may be generated such that the gas extracted by means of the extraction probe flows through the extraction probe at a predetermined flow velocity. The first pressure, the second pressure and/or any further pressure may be a gauge pressure.

The first pressure may be a pressure that the gas has in an extraction cross-section that forms the inlet opening. The second pressure may be a pressure that the gas has in an output cross-section that forms the outlet opening. The respective pressure may be a pressure measured at a measurement point, such as the inlet opening.

The inlet opening may form a first end portion of the extraction probe and/or the outlet opening may form a second end portion of the extraction probe.

The predetermined flow velocity may be determined based on the flow path and/or the measuring objective of the measuring device. The measuring objective of the measuring device may be a temporal measuring objective in which a measurement of the gas is performed within a predetermined time interval, respectively. This time interval may be 10 seconds, so that a measurement is made every 10 seconds. Alternatively or additionally, a one-time measurement may be required, which takes place within a predetermined period of time. This may be a quick measurement. Accordingly, the system, in particular the extraction probe may be configured such that the gas is extracted from the gas line and flows to the measuring device within 10 seconds, based on an appropriate flow velocity of the gas along the extraction probe and possible other lines and units of the system. If, due to the extraction probe and the pressure of the gas in the gas line, it is not possible for the gas to flow to the measuring device within the 10 seconds, a pump may be provided which provides and/or increases the pressure difference so that the flow velocity is generated such that the gas flows to the measuring device within the 10 seconds.

The flow path may comprise at least one circulation path and/or one measurement path, wherein the circulation path is a path that the gas extracted by means of the inlet opening flows from the inlet opening along the extraction line to the outlet opening, wherein the measurement path is a path that the gas extracted by means of the inlet opening flows from the inlet opening to that of the measuring device.

The inlet opening may be configured by the extraction cross-section for extracting the gas from the gas line and/or the outlet opening may be configured by the output cross-section for at least partially outputting the extracted gas into the gas line, wherein the extraction probe may be configured such that the extraction cross-section and/or the output cross-section are arrangeable perpendicular to the flow direction in the gas line. Consequently, the extraction cross-section and/or the output cross-section may each extend within a plane that is arranged perpendicular to the flow direction. Alternatively, the inlet opening and/or the outlet opening, in particular the extraction cross-section and/or the output section may each extend in a plane which is not perpendicular to the flow direction and/or is tilted relative to the flow direction. In particular, the extraction cross-section and/or the output cross-section may be configured and arrangeable in the gas line such that they are configured parallel to each other.

The extraction probe may be configured such that the extraction cross-section faces the gas flowing along the flow direction in the gas line, in particular such that the gas flowing in the gas line flows into the inlet opening due to the flow direction, and/or the output cross-section faces away from the gas flowing along the flow direction in the gas line, in particular such that the gas flowing in the extraction line flows out of the outlet opening in the direction of the flow direction and/or may be discharged. The outlet opening, in particular the output section, may be arrangeable such that it is oriented and/or opened in the direction of the flow direction, while the inlet opening, in particular the extraction cross-section, may be arrangeable such that it is oriented and/or opened against the flow direction. This structure makes it possible for the gas flowing in the gas line to encounter the inlet opening head-on due to its flow velocity, to flow into the inlet opening and to continue flowing through the extraction line. In the process, the gas may undergo a change in direction through the extraction line that is different from the flow direction, at least in sections. The gas flows via the extraction line at least partially to the discharge opening and may be output via the discharge opening. Thereby, the direction of the gas flowing in the extraction line at the discharge opening and/or an end portion of the extraction line comprising the discharge opening may be changeable such that the direction of the gas flowing in the extraction line is changeable in the direction of the flow direction, in particular is parallel to the flow direction. The gas flowing in the extraction line may be output by means of the discharge opening in the direction of the flow direction, in particular when the discharge opening faces away from the flow direction and is thus open in the direction of the flow direction. In combination with the arrangement of the extracting and/or output cross-sections perpendicular to the flow direction, in particular the extracting and/or output cross-section may be arranged 180° opposite to the flow direction.

The inlet and/or outlet opening, in particular the extracting and/or output cross-sections may be configured round, in particular circular.

The extraction probe may be configured arc-shaped and/or U-shaped at least in sections, the inlet and/or outlet openings forming in particular a respective end section of the extraction probe, in particular of the U-shaped extraction probe. The extraction probe may additionally or alternatively have elongated line sections.

The extraction line may have a plurality of line sections, in particular first to seventh line sections, and the extraction line may be configured and arrangeable in the gas line such that the line sections extend at least partially at an identical and/or different angle to the flow direction. The line sections may be fluidly connected to each other.

The extraction line may have a predetermined diameter. The predetermined diameter may correspond to a diameter of the circularly configured inlet and/or outlet openings, in particular the extracting and/or outlet openings, wherein the inlet and/or outlet openings have the predetermined diameter. Alternatively, the extraction line may have two, three or more different diameters.

The extraction line may be configured and at least partially arrangeable in the gas line in such a way that the first line section extends from the inlet opening at a first predetermined angle to the flow direction and a line section, in particular the seventh line section, extends from the outlet opening at a fourth predetermined angle to the flow direction, wherein the first and fourth angles are different and in particular differ by 90° from each other. The indicated dimensions in the form of the “°” sign of the angles may be degrees of arc or angular dimensions.

The extraction line may be configured and arrangeable in the gas line such that the second line section adjacent to the first line section extends perpendicular to the flow direction, and the sixth line section adjacent to the seventh line section extends perpendicular to the flow direction.

The extraction line may be configured and arrangeable in the gas line such that the third line section adjacent to the second line section extends at a second predetermined angle to the flow direction and the fifth line section adjacent to the sixth line section extends at a third predetermined angle to the flow direction, the second and third angles being different and in particular different from each other by 90°.

The extraction line may be configured and arrangeable in the gas line such that the fourth line section of the extraction line adjacent to the third and fifth line sections is configured in a U-shape and, in particular, the two longitudinal pieces of the U-shaped line section extend perpendicular to the flow direction. The U-shape may comprise a first longitudinal piece, configured in particular in a straight line, and a second longitudinal piece, configured in particular in a straight line, which are fluidically connected to each other by means of an arc-shaped piece.

The first angle may be between 10° and 80°, in particular 45°. The second angle may have between 100° and 170°, in particular 135°. The third angle may have between 10° and 80°, in particular 45°. The fourth angle may have between 100° and 170°, in particular 135°. The first and third angles may be identical. The second and fourth angles may be identical.

By means of the proposed extraction probe, a fast response time is ensured since the flow velocity of the gas in the gas line is used. In addition, a production of the extraction probe is simplified, since most components of it are not active or not to be controlled electronically and/or mechanically. Additionally, the risk of clogging is reduced with an appropriate slope of the line section comprising the inlet opening and a slope of the line section comprising the outlet opening.

The interface may be arranged at and/or in an arc-shaped section of the U-shaped extraction line, the fourth line section and/or an elongated line section.

The at least one measuring device is configured to measure the gas. Thereby, the at least one measuring device may be configured for measuring the quality of the gas. Further measuring devices of the system may measure further properties and/or parameters of the gas.

The system may comprise at least one pressure reducing device, a line configured to direct the gas from the interface to the pressure reducing device, the pressure reducing device being directed to reduce a pressure of the gas directed to the pressure reducing device to a predetermined pressure, and a line by means of which the gas at the reduced pressure is directed to the measuring device.

The gas line may be a natural gas line. The gas may be and/or comprise natural gas.

The inlet opening may be configured and arrangeable in the gas line such that the gas flowing in the gas line meets the inlet opening head-on.

The inlet and/or outlet openings may be configured and arrangeable in the gas line such that they are arrangeable at substantially the same height, in particular the same height in the gas line. The height extends along a height direction which is perpendicular to the flow direction. The height direction may be parallel to a direction of the gravitational force. In other words, the inlet and outlet openings may have substantially the same immersion depth, in particular the same immersion depth in the gas line, the immersion depth also extending along the height direction, but compared to the height in the other direction along the height direction.

The extraction probe may be configured in the form of a line and/or a pipe. The gas extracted by means of the extraction probe may be directed by means of at least one pipe and/or at least one line from the interface to the pressure reducing device and by means of another pipe and/or another line from the pressure reducing device to the measuring device.

The extraction probe may have two, three or more interfaces, and/or the system may comprise two, three or more pressure reducing devices and/or measuring devices, wherein in particular by means of the two, three or more interfaces the gas is at least partially directed from the extraction line to the two, three or more pressure reducing devices and/or measuring devices.

The inlet and/or outlet opening may be arrangeable in the gas line in such a way that they are arranged along an arrangement direction extending parallel to the flow direction.

The extraction probe may have at least one valve, in particular a double-block-and-bleed valve, upstream and/or downstream of the interface of the extraction line, in particular downstream along the extraction line as viewed after the last interface of the two, three or more interfaces and/or upstream of the first interface of the two, three or more interfaces. The double block-and-bleed valve may have two shut-off valves (block) and one bleed valve (bleed). If the block and bleed valve fails to close in the event of a failure, the second block and bleed valve prevents uncontrolled media leakage or contamination of the process from the outside.

The system may comprise a housing configured such that the at least one pressure reducing device and/or the at least one measuring device are incorporated in the housing, wherein the housing is configured such that the extraction probe, in particular the extraction line is incorporated at least partially in the housing. In particular, the arc-shaped section and/or the fourth line section may be incorporated at least partially into the housing.

The system may further comprise at least one pump unit configured to at least partially provide the pressure difference and/or amplify the pressure difference. The pump unit may be configured to generate the pressure difference such that the predetermined flow velocity is generated.

The extraction probe may further comprise a line housing, which comprises the extraction line at least in sections. In this context, the line housing may be configured in such a way that the line housing may be inserted at least in sections into the gas line, in particular a corresponding opening of the gas line. In particular, the line housing may be arrangeable in the gas line and/or the opening such that the line housing fluidically seals with the gas line and/or the opening. Further, the line housing may be configured such that it additionally or alternatively comprises at least the U-shaped section of the extraction probe. The line housing may be incorporated in and/or connected to the housing, at least in sections.

The extraction probe may be configured in such a way that a stagnation pressure of the gas may be generated at the inlet opening and a dynamic pressure may be generated at the outlet opening, in such a way that the pressure difference between the inlet and outlet openings may be generated. The first pressure, in particular the stagnation pressure may be generated higher than the second pressure, in particular the dynamic pressure.

The objective is solved according to a second aspect by an arrangement comprising a system according to the first aspect and the gas line, wherein at least the inlet opening and the outlet opening are arranged in the gas line.

### BRIEF DESCRIPTION OF THE DRAWINGS

Preferred embodiments are exemplified with reference to the accompanying figures. It shows:

FIG. 1 a system for measuring the quality of a gas flowing in a gas line; and

FIG. 2 an embodiment of an extraction probe for such a system.

In the figures, identical or substantially functionally identical or similar elements are designated with the same reference signs.

### DETAILED DESCRIPTION OF ILLUSTRATIVE EMBODIMENTS

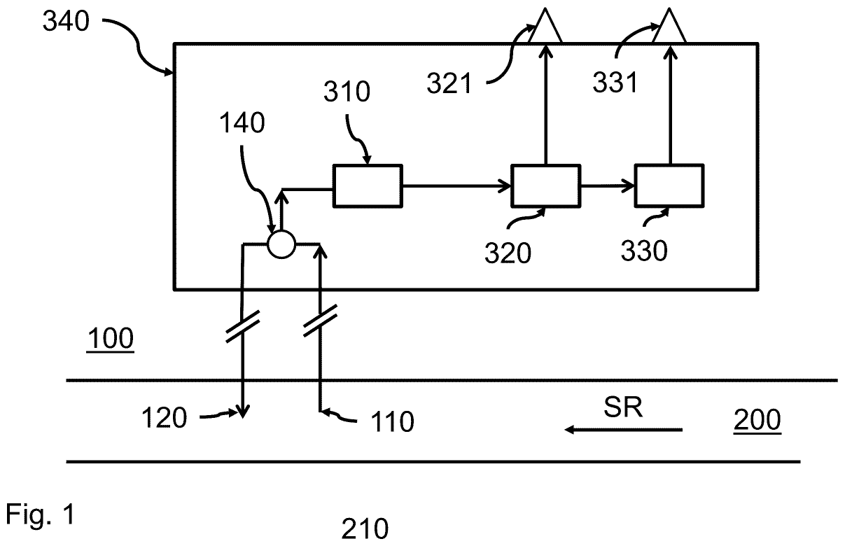

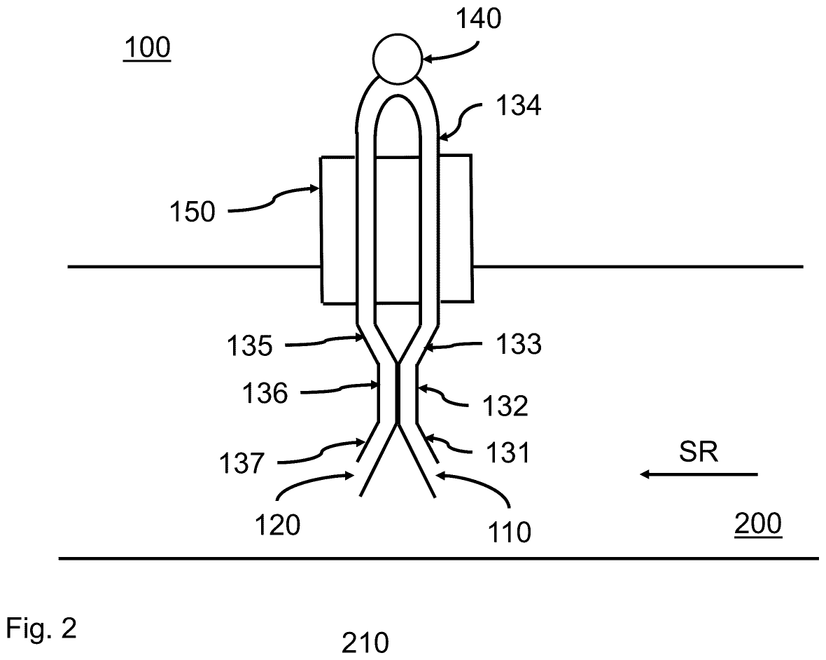

FIG. 1 shows a system for measuring the quality of a gas flowing in a gas line 200, the gas flowing along a flow direction SR at a predetermined pressure and flow velocity. The system includes an extraction probe 100 for extracting gas from the gas line 200, the extraction probe 100 being further configured for at least partially returning the gas to the gas line 200. The arrows in FIGS. 1 and 2 may be lines and/or pipes configured to direct the gas. The lines and/or pipes may fluidically connect the respective units of the system. The arrows have arrow directions indicating a respective flow direction of the gas along the lines and/or pipes. The system includes an optional housing 340 that incorporates an optional pressure reducing device 310, an optional bypass 320, and a measuring device 330. Further, the extraction probe 100 is at least partially incorporated into the housing 340.

The extraction probe 100 includes an interface 140 that is fluidly connected to the pressure reducing device 310. The extraction probe 100 may include two, three, or more interfaces, although only one is shown in FIG. 1. In this regard, a section of the extraction probe 100 at and/or in which the interface 140 is arranged is incorporated into the housing 340. By means of the interface 140, a predetermined portion of the gas extracted by means of the extraction probe 100 may be extracted and directed to the pressure reducing device 310.

Due to the high pressure of the gas in the gas line and thus also in the extraction probe 100, it may be necessary for measuring the quality of the gas to reduce the pressure of the gas by means of the pressure reducing device 310. This may depend on the type of measuring device and/or the type of measurement. The gas may then be directed at reduced pressure to the optional bypass 320 or directly to the measuring device 330. The bypass 320 is configured to forward a portion of the low pressure gas further to the measuring device 330, and the remaining portion to the environment via the outlet 321. The bypass 320 may be configured to increase a flow rate of the gas through the bypass 320, the measuring device 330, a bypass outlet 321, and/or a measuring device outlet 331, in particular upon falling below a predetermined pressure difference between a pressure of the gas along a respective line upstream of and/or at the bypass 320 and a pressure of the gas downstream of the bypass 320 at and/or at at least one of the measuring device 330, the bypass outlet 321, and/or the measuring device outlet 331.

The measuring device 330 is configured to measure the extracted gas, in particular a quality of the gas. For this purpose, the measuring device 330 may comprise a gas chromatograph and an inlet for a carrier gas. The housing 340 may further comprise a unit providing the carrier gas. The gas measured by the measuring device 330 may be discharged to the environment by means of the outlet 331.

In order to minimize environmental impact, the amount of gas released to the environment should be minimized. Further, it would be advantageous to have to extract as little gas as possible from the gas line for measuring the quality. Likewise, it is desirable that a distance between an extraction point of the gas line 200 and the system may be flexibly designed, in particular may have several meters.

These and other advantages arise with the extraction probe 100 in that it has an inlet opening 110 and an outlet opening 120, both of which are arranged in the gas line 200, the outlet opening 120 being arranged downstream along the flow direction SR of the gas flowing in the gas line 200, in particular downstream of the inlet opening 110. The inlet opening 110 is arranged upstream, in particular upstream of the outlet opening 120 along the flow direction SR of the gas flowing in the gas line 200. The inlet and outlet openings 110, 120 respectively form end sections of the extraction probe 100, which are fluidically connected to each other by means of an extraction line of the extraction probe 100.

In this regard, the inlet opening 110 is configured by an inlet cross-section extending within a plane perpendicular to the flow direction SR. The outlet opening 120 is configured by an output cross-section that is configured parallel to said plane. Here, the inlet opening 110 faces the gas flowing in the gas line 200 and the outlet opening 120 faces away from the flowing gas. Due to this structure of the extraction probe 100, the gas flows from the gas line via the inlet opening 110 into the extraction probe. This causes a stagnation pressure to form at the inlet opening 110, which is higher than the pressure of the gas in the gas line. Further, a dynamic pressure is formed at the outlet opening 120 which is lower than the pressure of the gas in the gas line, in particular lower than the stagnation pressure. Consequently, there is a pressure difference between the inlet and outlet openings, resulting in a flow or flow velocity of the gas along the extraction probe 100, in particular the extraction line. The gas flows along the extraction line at this flow velocity and may be at least partially extracted for measurement purposes by means of the at least one interface 140. The remaining portion of the gas continues to flow to the outlet opening 120 and may consequently be fed back into the gas line 200.

In FIG. 1, it is indicated by the respective double lines running diagonally in FIG. 1 downstream after the inlet opening 110 and upstream before the outlet opening 120 that these line sections, in particular of the extraction line may have a predetermined length. Likewise, the extraction probe 100, in particular the extraction line is not limited to this form of line. As already mentioned at the beginning, the pressure of the gas in the gas line 200 is used in a particularly advantageous manner in order to be able to extract gas from the gas line 200 by means of the extraction probe 100, to direct this gas at a predetermined flow velocity along the extraction probe 100, in particular the extraction line, and finally to output it again at least partially via the outlet opening 120. Depending on the pressure of the gas in the gas line 200 and/or a structure of the extraction probe 100, in particular the extraction line, the gas may flow in the extraction probe 100 at a predetermined flow velocity. Depending on the application of the system and/or a measuring objective of the measuring device 330 and/or further aspects, such as a safe distance to the gas line 200, the gas may be extracted from the gas line 200 and at least partially returned without further means by means of the extraction probe 100. Consequently, a distance between the gas line 200 and the housing 340 may be several meters. A design of the extraction probe 100, in particular of the extraction line may advantageously be determined by a flow simulation. Possible parameters for this simulation may include the pressure of the gas in the gas line 200, a diameter of the extraction line and/or the inlet and outlet openings 110, 120, and an anticipated distance to the housing 340 and/or the interface 140.

If the pressure difference between the stagnation pressure and the dynamic pressure and the resulting pressure at the inlet opening and the resulting pressure at the outlet opening is insufficient, the system may further comprise a pump configured to increase the pressure difference to a predetermined pressure difference such that a desired flow velocity of the gas along the extraction probe 100 is generated.

Further, the extraction probe 100 may comprise one, two or more valves, in particular double block-and-bleed valves, arranged along the extraction line. At least one such valve may be arranged between two interfaces of the extraction probe 100.

FIG. 2 shows an embodiment of an extraction probe 100. The extraction probe 100 has an inlet opening 110 for extracting gas from the gas line 200. Referring to FIG. 2, the inlet opening 110 is configured and arrangeable in the gas line 200 such that the gas meets the inlet opening 110 head-on. Accordingly, an extraction cross-section of the inlet opening 110 may be configured perpendicular to the flow direction SR. This may provide for maximum extracting of gas by means of the extraction opening 110.

The extraction probe 100 further comprises an outlet opening 120, which is arrangeable downstream along the flow direction SR after the inlet opening 110 in the gas line 200. By means of the outlet opening 120, at least part of the gas extracted by means of the inlet opening 110 may be fed back into the gas line 200. In this context, the inlet opening and outlet opening may be arrangeable in the gas line 200 in such a way that they are arranged along an arrangement direction extending parallel to the flow direction SR. In this context, partial means that a portion of the gas extracted by means of the inlet opening 110 is directed to the pressure reducing device 310 via the interface 140.

For this purpose, the inlet opening 110 and the outlet opening 120 are fluidically connected by means of an extraction line comprising line sections 131 to 137. In particular, the extraction probe 100 may be referred to as a “loop” or circuit extraction probe 100, since, as shown in FIG. 2, the gas is extracted by means of the inlet opening 110, guided along a loop or circuit to the outlet opening 120 via the line sections 131-137. By means of the interface 140, a portion of the gas may be extracted at a fourth line section 134 and further directed to the pressure reducing device 310. The remaining gas may continue to the outlet opening 120 and be returned to the gas line 200.

Advantageously, the pressure and flow velocity of the gas in the gas line 200 is used as previously described to cause the gas to flow through the inlet opening 110 along the extraction line to the outlet opening 120.

The extraction line includes line sections 131 to 137. The first line section 131 is a first end section of the extraction line and comprises the inlet opening 110. Thereby, the first line section 131 is configured and arrangeable in the gas line 200 such that it has a predetermined angle between 10° and 80°, in particular 45° to the flow direction.

The angle between a line section and the flow direction SR may be an angle between an outer surface, an inner surface and/or a main extension direction of the line section and the flow direction SR. Referring to FIG. 2, the extraction line, in particular the line sections 131-137 have a predetermined diameter.

Further, one or more of the line sections 131-137 may have a circular, in particular, a circular cross-section. The line sections 131-133 and 135-137 may have a cylindrical shape. The cylindrical shape may extend along the main extension direction with a predetermined cylinder height and perpendicular to the main extension direction with a predetermined cylinder radius. The line section 134 is U-shaped, shown in the form of an inverted U according to FIG. 2. Here, the line section 134 has two elongated longitudinal pieces extending perpendicular to the flow direction SR, which are fluidically connected to the line sections 133, 135. These longitudinal pieces may be configured in a cylindrical shape. Further, the line section 134 comprises an arc-shaped piece between the two longitudinal pieces, wherein the interface 140 is arranged at and/or in the arc-shaped piece. The interface 140 may be configured to fluidically seal with the extraction line and/or at least partially forward gas to the pressure reducing device 310.

Referring to FIG. 2, a second line section 132 is fluidly connected to the first line section 131 and extends perpendicular to the flow direction SR. Fluidly connected to the second line section 132 is a third line section 133 which is arranged at a predetermined angle, in particular between 100° and 170° to the flow direction SR. Viewed along the extraction line, the line sections 135-137 are arranged mirror-inverted with respect to the line sections 131-133, the mirroring being along an axis extending perpendicular to the flow direction SR. The line sections 131-137 are arranged in series and fluidically connected to each other. By means of the arrangement of the line sections 131-137 shown in FIG. 2, a pressure loss along the extraction probe 100 is particularly low.

Further, FIG. 2 shows a line housing 150 which comprises the extraction line at least in sections. In this case, the line housing 150 is configured in such a way that the line housing 150 may be inserted at least in sections into the gas line 200 and/or seals fluidically therewith. In particular, the line housing 150 may be arrangeable in the gas line 200 such that the line housing 150 fluidically seals with a corresponding opening of the gas line 200. Further, the line housing 150 may be configured such that it additionally or alternatively comprises at least the U-shaped section of the extraction probe 100.

What is claimed is:

1. System for measuring a gas flowing in a gas line, comprising:
an extraction probe having an inlet opening for extracting a gas flowing in a gas line, an outlet opening for at least partially outputting the extracted gas into the gas line, and an extraction line fluidically connecting at least the inlet opening and the outlet opening,
wherein the extraction probe is configured such that at least the inlet opening and the outlet opening are arrangeable in the gas line,
the extraction probe being configured in such a way that the outlet opening is arrangeable downstream of the inlet opening along a flow direction of the gas flowing in the gas line;
at least one measuring device for measuring the gas,
the extraction line having at least one interface via which the gas is directable at least partially to the measuring device.

2. System according to claim 1,
wherein the inlet opening is configured and arrangeable in the gas line such that the gas flowing in the gas line is extractable from the gas line by means of the inlet opening due to a flow velocity of the gas in the direction of the flow direction.

3. System according to claim 1,
wherein the extraction probe is configured such that a pressure difference between a first pressure of the gas extracted by means of the extraction probe at the inlet opening and a second pressure of the gas directed by means of the extraction line is generatable at the outlet opening,
wherein the pressure difference is generatable in such a way that the gas extracted by means of the extraction probe flows through the extraction probe at a predetermined flow velocity.

4. System according to claim 3,
wherein the predetermined flow velocity is determined based on a flow path and/or a measuring objective of the measuring device,
wherein the flow path comprises a circulation path and/or a measurement path,
wherein the circulation path is a path along which the gas extracted by means of the inlet opening flows from the inlet opening along the extraction line to the outlet opening,
wherein the measurement path is a path that the gas extracted by means of the inlet opening flows from the inlet opening to that of the measuring device.

5. System according to claim 1,
wherein the inlet opening is configured by an extraction cross-section for extracting the gas from the gas line and/or the outlet opening is configured by an output cross-section for at least partially outputting the extracted gas into the gas line,
wherein the extraction probe is configured such that the extraction cross-section and/or the output cross-section are arrangeable perpendicular to the flow direction in the gas line.

6. System according to claim 1,
wherein the extraction probe is configured such that an extraction cross-section for extracting the gas from the gas line and/or the outlet opening faces the gas flowing along the flow direction in the gas line and/or an output cross-section for at least partially outputting the extracted gas into the gas line faces away from the gas flowing along the flow direction in the gas line.

7. System according to claim 1,
wherein the extraction probe comprises a double block-and-bleed valve, upstream and/or downstream of the interface of the extraction line.

8. System according to claim 1, further comprising:
at least one pressure reducing device;
a line configured to direct the gas from the interface to the pressure reducing device,
the pressure reducing device being configured to reduce a pressure of the gas directed to the pressure reducing device to a predetermined pressure;
a line by means of which the gas at the reduced pressure is directed to the measuring device.

9. System according to claim 1, further comprising:
a housing configured such that the pressure reducing device and/or the measuring device are incorporated into the housing,
wherein the housing is configured such that the extraction probe and the extraction line are at least partially incorporated into the housing.

10. System according to claim 1, further comprising:
at least one pump unit configured to at least partially provide a pressure difference between the inlet and outlet openings.

11. System according to claim 10,
wherein the pump unit is arranged along the extraction line downstream of the interface.

12. System according to claim 1,
wherein the extraction probe is configured to generate a stagnation pressure of the gas at the inlet opening and a dynamic pressure at the outlet opening, such that a pressure difference between the inlet and outlet openings is generated.

13. System according to claim 1,
wherein the extraction line comprises two, three or more interfaces, and/or
wherein the system comprises two, three or more pressure reducing devices and/or measuring devices,
wherein by means of the two, three or more interfaces the gas is at least partially directed from the extraction line to the two, three or more pressure reducing devices and/or measuring devices.

14. Arrangement comprising a system according to claim 1 and a gas line, wherein at least the inlet opening and the outlet opening are arranged in the gas line.
